(19) United States
(12) Patent Application Publication
(10) Pub. No.: US 2023/0245786 A1
(43) Pub. Date: Aug. 3, 2023

(54) METHOD FOR THE PROGNOSIS OF A DESEASE FOLLOWING UPON A THERAPEUTIC TREATMENT, AND CORRESPONDING SYSTEM AND COMPUTER PROGRAM PRODUCT

(71) Applicant: Aizoon S.r.l., Torino (TO) (IT)

(72) Inventors: Lorenzo MANGANARO, Torino (TO) (IT); Gianmarco SABBATINI, Torino (TO) (IT); Selene BIANCO, Torino (TO) (IT); Francesca CIPOLLINI, Torino (TO) (IT); Davide COLOMBI, Torino (TO) (IT); Shaji VATTAKUNNEL, Torino (TO) (IT); Paolo FALCO, Torino (TO) (IT)

(21) Appl. No.: 18/095,931

(22) Filed: Jan. 11, 2023

(30) Foreign Application Priority Data
Feb. 2, 2022 (IT) 102022000001817

(51) Int. Cl. G16H 50/70; G16H 10/60

(52) U.S. Cl. CPC G16H 50/70; G16H 10/60

(57) ABSTRACT

Solutions for the prognosis of a given disease provided. A classifier is trained for estimating the index as a function of the respective set of features using a training dataset. For prognosis, further patient omics data is received. A verification dataset is generated by forming pairs of patients by combining the patient with the reference patients and determining, for each pair, a respective set of features. Estimation is performed through the classifier, for each pair of patients, of a respective index to generate an orderly list of the estimated indices. At each position in the list a respective parameter that indicates a probability is calculated. The position with the highest probability is selected and the disease-free survival time of the patient is estimated as a function of disease-free survival time of the reference patient in the selected position.

### TECHNICAL FIELD

The embodiments of the present disclosure relate to techniques for estimating disease-free survival following upon a therapeutic treatment, which may be used for prognosis of the desease.

### BACKGROUND

It is deemed that genetic profiles can be linked to prognosis. Such prognosis is frequently quantified through a quantity that indicates the time of disease-free survival (DFS), typically from cancer, after a particular treatment.

With the invention and recent reduction in cost of next-generation sequencing (NGS) technology, a large amount of omics data has become progressively available for biocomputational analyses. In particular, NGS technology is an in vitro process of analysis that comprises sequencing in parallel and that enables sequencing of large genomes over a very restricted time. The term “omics” refers to data that identify genomics, transcriptomics, proteomics, metabolomics, and/or metagenomics.

In the last few years, there has thus been a growing interest in the development of machine-learning (ML) models, which are able to decode these correlations, for example for estimating the evolution of the DFS parameter or a similar index as a function of the omics data. For example, in this context it is possible to cite the European patents EP 1 977 237 B1, EP 2 392 678 B1, EP 2 836 837 B1, EP 3 237 638 B1, or EP 2 700 038 B1.

For instance, frequently models that estimate DFS accompany the estimates with a measurement of probability: the decrease in the probability of disease-free survival as a function of time is known as survival curve. For example, to estimate disease-free survival time of a patient, the machine-learning model may comprise a parameterized mathematical function, such as an artificial neural network, configured for estimating the time of disease-free survival of a given patient as a function of the omics data obtained for the patient in question, or as a function of a plurality of features drawn from these omics data. Consequently, by acquiring a training dataset that comprises the omics data of a plurality of patients and the respective disease-free survival times, a training algorithm may modify, typically through an iterative process, the parameters of the mathematical function in such a way as to reduce the difference between the estimate of the disease-free survival time and the respective data of the dataset. Consequently, once the learning model has been trained, the mathematical function can provide an estimate of the disease-free survival time for a patient as a function of the respective omics data of the patient.

Instead of directly estimating the DFS, also other parameters indicative of the DFS may be estimated. For example, the article by Tong Li, et al, “Deep learning based feature-level integration of multi-omics data for breast cancer patients survival analysis”, vol. 20, no. 1, 1 Dec. 2020 (2020-12-01), XP055957087, DOI: 10.1186/s12911-020-01225-8, discloses a solution wherein a machine learning method, in particular an autoencoder, is used as a feature extraction algorithm in order to generate a set of hidden features. The hidden features are then used to perform a multi-class classification and a survival analysis. Specifically, the survival analysis uses a neural network, which estimates a “hazard” based on the hidden features, wherein the neural network is trained with a negative log partial likelihood loss taking into account the survival times of the patients. A similar solution for estimating a hazard/risk score is disclosed in the article by Chai Hua, et al., “Integrating multi-omics data through deep learning for accurate cancer prognosis prediction”, bioiv, 6 May 2021 (2021-May 2006), XP055957085, D2 DOI:10.1101/807214.

Thus, such a hazard score is in some way indicative of the survival time. For example, the articles by Tong Li and Chai Hua uses a concordance index (C-index) in order to evaluate how well the estimated survival risk/hazard aligns with the actual survival times. In this respect, as also disclosed in document Uno Hajime, et al: “On the C-statistics for evaluating overall adequacy of risk prediction procedures with censored survival data”, STATISTICS IN MEDICINE, vol. 30, no. 10, 10 May 2011 (2011-May-10), pages 1105-1117, XP055957193, US, ISSN: 0277-6715, DOI: 10.1002/sim.4154, such C-statistics are routinely used in the medical literature to assess and quantify the capacity of an estimated risk score in discriminating among subjects with different event times, e.g., via a rank correlation measure.

One of the main problems in this field is that the training dataset typically includes a limited number of samples, especially if compared to the enormous number of features that need to be analyzed for an effective prognosis. In fact, clinical trials typically involve from a few hundreds up to 1500/2000 patients, whereas NGS data may potentially provide as many features as are the human genes: approximately 20 000 in the case of the transcriptome if the analysis is limited to the genes that encode proteins, and a factor of two to three times more if also the non-encoding genes are considered. From the standpoint of machine learning, this problem of dimensionality (generally known as “curse of dimensionality”) prevents the models from learning, leading to an over-adaptation with respect to the training dataset and to the loss of the capacity of generalization, since there are not enough samples for extrapolating a general behaviour.

### SUMMARY

Various embodiments of the present disclosure regard solutions for prognosis, in particular with reference to the estimate of the disease-free survival time following upon a therapeutic treatment. According to one or more embodiments, this object is achieved through a method having the distinctive elements set forth specifically in the ensuing claims. The embodiments moreover regard a corresponding device, as well as a corresponding computer program product that can be loaded into the memory of at least one computer and comprises portions of software code for executing the steps of the method when the product is run on a computer. As used herein, reference to such a computer program product is intended as being equivalent to reference to a computer-readable means containing instructions for controlling a processing system in order to co-ordinate execution of the process. Reference to “at least one computer” is clearly intended to highlight the possibility of the present disclosure being implemented in a distributed/modular way.

The claims form an integral part of the technical teaching of the description provided herein.

As mentioned previously, various embodiments of the present disclosure regard solutions for prognosis of a desease following upon a therapeutic treatment of the desease, such as a particular type of tumour or cancer. As explained previously, in various embodiments, machine-learning solutions are used for this purpose.

In particular, in various embodiments, during a learning step, a computer receives a dataset comprising a plurality of reference patients who have undergone a therapeutic treatment of the same desease, where the dataset comprises for each reference patient respective omics data and data that indicate a disease-free survival time of the respective reference patient after the respective therapeutic treatment of the desease. For instance, in various embodiments, the omics data comprise transcriptomics data, for example obtained via NGS.

In various embodiments, the computer then generates, for each reference patient, respective pre-processed data, e.g., via an analysis of the principal components of the respective omics data, and stores the respective mapping or combination rules used to generate the pre-processed data as a function of the respective omics data. Optionally, the computer may also normalize the omics data, and/or scale the omics data via a nonlinear function.

In various embodiments, the computer then generates a training dataset to form pairs of reference patients between a respective first reference patient and a respective second reference patient and to determine, for each pair of reference patients, a respective set of features that is calculated via a measurement of distance between the pre-processed data of the respective two reference patients and a respective index that indicates which of the respective two reference patients has a longer disease-free survival time. For instance, for this purpose, the computer may set the index at a first value to indicate the fact that the respective first reference patient has a disease-free survival time that is longer than the disease-free survival time of the respective second reference patient, or else at a second value to indicate the fact that the respective first reference patient has a disease-free survival time that is shorter than the disease-free survival time of the respective second reference patient.

In various embodiments, the computer then trains a classifier configured for estimating the index as a function of a respective set of features that is calculated for the pre-processed data of two patients, using for this purpose the training dataset. For instance, the classifier may comprise at least one of the following: a k-nearest neighbour classifier, an artificial neural network, such as a network of the multilayer-perceptron type, a support vector machine, a Gaussian-process classifier, decision trees, random forests, quadratic discriminant analysis, and/or Gaussian naive Bayes classifiers. In various embodiments, the computer may also select just a subset of the features via a feature-selection method, such as LASSO, and train the classifier in such a way as to estimate the index as a function of the respective subset of features calculated for the pre-processed data of two patients.

Accordingly, compared to the prior art solutions, such as the articles by Tong Li and Chai Hua, the classifier is not trained via the omics data of each individual reference patient, but the input features of the classifier correspond to a distance measurement between the data of two patients. Accordingly, assuming a dataset with n reference patients, the prior-art solutions may use a training dataset with up to n training records, while the solutions disclosed herein may use a training dataset with up to n(n−1) training records, thus significantly increasing the available training data. However, this also implies that the trained classifier does not estimate directly the disease-free survival time (or a similar value such as the hazard or survival risk), but the classifier just estimates an index that indicates which of two patients has a longer disease-free survival time, and a further post-processing is required in order to estimate the disease-free survival time.

Consequently, during a prognosis step, the computer may receive for a (further) patient respective omics data and generate for the patient respective pre-processed data using the mapping or combination rules. In various embodiments, the computer then generates a verification dataset to form pairs of patients by combining the patient with a plurality of reference patients (and preferably with each reference patient) and by determine, for each pair of patients, a respective set of features calculated via the measurement of distance between the pre-processed data of the patient and the pre-processed data of the respective reference patient. Consequently, at this point, the computer may estimate, for each pair of patients, a respective index, supplying to the trained classifier the set of features of the respective pair of patients.

As mentioned before, compared to the prior-art solutions, the classifier is not used to estimate directly the disease-free survival time (or a similar value such as the hazard or survival risk) for the patient based on the omics data of the patient. For example, in the articles by Tong Li and Chai Hua, the classifier provides an individual and absolute risk score for each patient. In this respect, the performance of such prior-art classifiers may be estimated through the C-index values, wherein the C-index represents the fraction of all pairs of individuals whose predicted survival times are correctly ordered based on the estimated hazard, i.e., the C-Index is a performance index of the classifier. Conversely, in the present invention, the classifier estimates for each patient pair an index, which essentially represents a relative score. Accordingly, in the present invention, these indices have to be recalculated for each new patient for a set of reference patients, and just provide a relative information for the pair of patients. However, as will be described in greater detail in the following, the disease-free survival time of the patient may be estimated based on the disease-free survival times of the set of reference patients and the set of estimated indices. Accordingly, also the performance of the present solution could be evaluated by calculating a C-Index for the disease-free survival times estimated by the present solution.

Specifically, in various embodiments, the computer generates in parallel or subsequently a list of the estimated indices, where the list is ordered according to the disease-free survival time of the reference patients of the pairs of patients. In various embodiments, the computer then computes, for each position of a plurality of positions of the list (and preferably for all the positions), a respective parameter that indicates the probability of the patient having a disease-free survival time longer than the disease-free survival time of the reference patient in the respective position, but shorter than the disease-free survival time of the reference patient in the next position. For instance, for this purpose, the computer may generate, for each position, a respective pattern to be verified by selecting the index of the list in the respective position, and a first number of indices of the list before the position, and/or a second number of indices of the list after the position. Then, the computer obtains for each pattern to be verified a respective reference pattern, where the reference pattern corresponds to pattern of indices in the case where the position corresponds to the position where switching occurs between a sequence of the first number of the first value and a sequence of the second number of the second value, and computes the value of the parameter associated to the position via a measurement of similarity or a measurement of distance between the respective pattern to be verified and the respective reference pattern. For instance, in various embodiments, each pattern to be verified corresponds to the sequence of the estimated indices of the list, and the reference pattern associated to a given position has all the indices up to the position set at the first value and all the indices after the position set at the second value.

Consequently, in various embodiments, the computer may select the position for which the respective parameter indicates the highest probability, and estimate the disease-free survival time of the patient as having a value comprised between the disease-free survival time of the reference patient in the selected position and the disease-free survival time of the reference patient in the position that follows the selected position.

### BRIEF DESCRIPTION OF THE DRAWINGS

The embodiments of the present disclosure will now be described with reference to the annexed drawings, which are provided purely by way of non-limiting example and in which:

FIG. 1 shows a flowchart of a method and corresponding operation of a computer configured for estimating the disease-free survival time of a patient via a learning step and a prognosis step;

FIG. 2 shows an example of a processing system capable of implementing the operation of FIG. 1;

FIGS. 3 and 4 show examples of data used in FIG. 1;

FIG. 5 shows an embodiment of the learning step of FIG. 1;

FIGS. 6, 7, and 8 show examples of data used in FIG. 5;

FIG. 9 shows an embodiment of the prognosis step of FIG. 1;

FIGS. 10, 11, 12, and 13 show examples of data used in FIG. 9; and

FIG. 14 shows a detail of operation of the embodiment of FIG. 9.

### DETAILED DESCRIPTION

In the ensuing description numerous specific details are provided in order to enable an in-depth understanding of the embodiments. The embodiments may be implemented without one or various specific details, or with other methods, components, materials, etc. In other cases, well-known operations, materials, or structures are not represented or described in detail so that the aspects of the embodiments will not be obscured.

Reference throughout the ensuing description to “an embodiment” or “one embodiment” means that a particular characteristic, distinctive element, or structure described with reference to the embodiment is comprised in at least one embodiment. Thus, use of phrases such as “in an embodiment” or “in one embodiment” in various points of this description do not necessarily refer to one and the same embodiment. Moreover, the details, characteristics, distinctive elements, or structures may be combined in any way in one or more embodiments.

The references used herein are provided merely for convenience and do not define the scope or meaning of the embodiments.

As explained before, to enable a more accurate prognosis, recently also omics data are taken in consideration, which are processed by means of a machine-learning algorithm. For instance, in various embodiments, the present solution is used for estimating the disease-free survival time for patients affected by non-small cell lung cancer (NSCLC), who have undergone a surgical operation and have obtained complete removal of the neoplasm. However, the solution proposed herein may be used for estimating the disease-free survival time also for other diseases, and in particular for other types of cancers. For instance, such an estimate may represent auxiliary information fundamental for oncologists, who can make better-informed decisions, for example vary the frequency of the follow-up checks as a function of the time estimated, for instance increase the frequency of the checks for patients with an estimated time that is short.

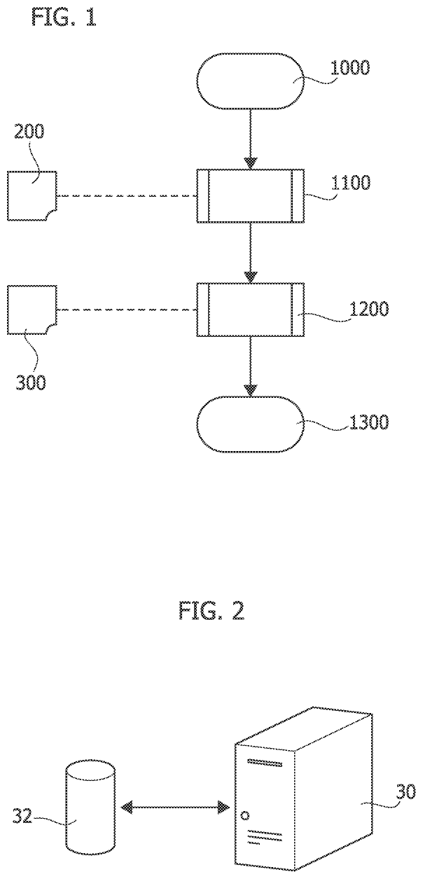

FIG. 1 shows an embodiment of operation of a computer 30 configured for estimating the disease-free survival time of a given patient (output datum) as a function of the respective values of the set of parameters (input data).

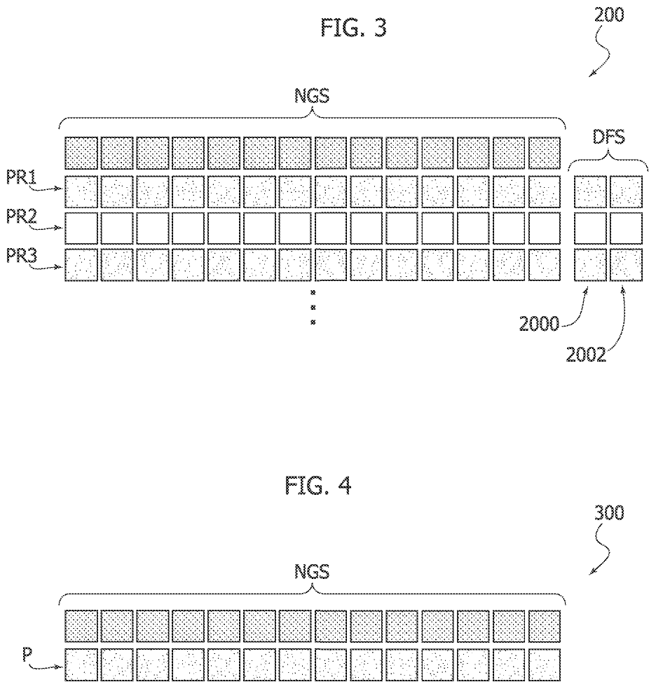

For instance, as illustrated in FIG. 2, the computer 30 may be implemented with any processing system, possibly also in distributed form, and may comprise, for example, a computer, a smartphone or tablet, and/or a remote server. Consequently, operation of the computer 30 may be implemented via software code executed by one or more computers. For instance, in this case, the dataset 200 may be stored in one or more databases 32 managed by the computer 30.

In the embodiment considered, after an initial step 1000, the computer 30 trains, in a step 1100, a machine-learning algorithm using a training dataset 200 that comprises omics data for a plurality of patients. Consequently, in a step 1200, the computer 30 may use the trained algorithm for estimating the disease-free survival time of a patient as a function of the respective omics data 300, and the process terminates in an end step 1300.

As explained previously, the omics data 200 and 300 may comprise NGS transcriptomics data (RNA-seq), where the gene expression is expressed, for example, in transcripts per million (TPM). For instance, some examples of these data are made available by The Cancer Genome Atlas (TCGA) and may be freely downloaded from many sources, such as https://gdac.broadinstitute.org/ or https://www.cbioportal.org/. For instance, with reference to NSCLC, a dataset 200 may be used that comprises the lung-adenocarcinoma (LUAD) TCGA databases and/or the lung-squamous-cell-carcinoma (LUSC) TCGA databases, which currently represent the main histological subtypes of NSCLC.

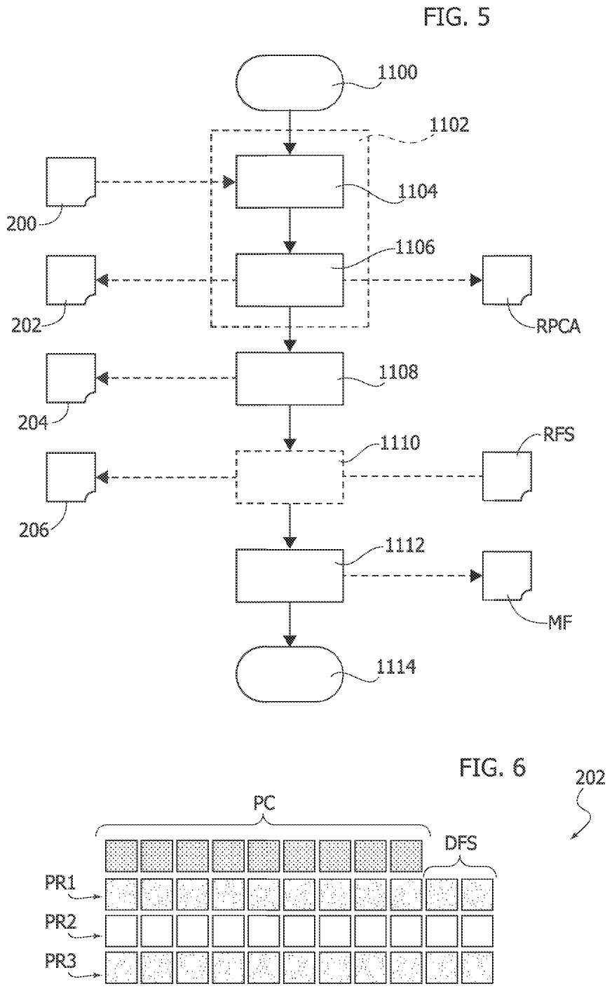

Consequently, as illustrated in FIG. 3, the dataset 200 corresponds to a table, list, or matrix of data that comprises the values for a set of variables for a plurality of reference patients PR. In particular, with reference to data NGS, each variable corresponds to the gene expression of a particular gene. For instance, the dataset 200 may correspond to a data matrix, in which each row of the matrix represents a (sample) reference patient PR, for example patients PR₁, PR₂, PR₃, etc., and each column represents the gene expression of a particular gene, for example expressed in TPM.

Moreover, each sample/patient of the dataset 200 has, associated thereto, data DFS that identify the respective disease-free survival time. For instance, in various embodiments, the disease-free survival time DFS of a given patient is identified via a right-censored survival datum, i.e., a pair made up of a Boolean value 2000 and a numeric value 2002, where the Boolean value 2000 indicates whether a relapse of the tumor has been noted for that particular patient (TRUE) or whether, at the date of the last check, no relapse has been noted (FALSE). Moreover, the numeric value 2002 indicates the moment of the relapse in the case where the Boolean is TRUE, or the date of the last check in the case where the Boolean is FALSE. If a patient dies without a tumor for independent reasons, he or she is considered “censored”, in which case the Boolean 2000 is set at FALSE, and the numeric value 2002 indicates the moment of death. For instance, this type of coding is used by the TCGA database. However, the datum DFS may be identified also via other parameters, and/or a number of parameters, and/or the numeric value 2002 may not identify a date, but directly a period with respect to the date of the operation, for example, a period that has elapsed up to relapse, a period that has elapsed up to the last check, or a period that has elapsed up to death.

Consequently, as shown in FIG. 4, the data 300 of a patient P comprise the data NGS, and the computer 30 should estimate, in step 1200, a respective disease-free survival time as a function of these data 300.

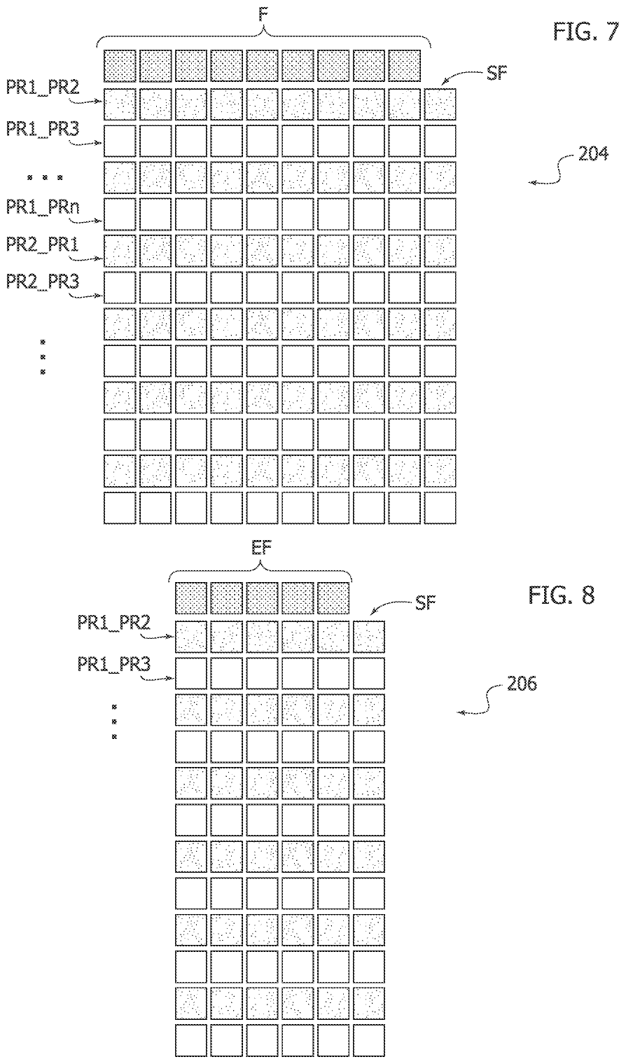

FIG. 5 shows a possible embodiment of the learning step 1100. Once the learning step 1100 has been started, the computer 30 obtains the training dataset 200. In various embodiments, the computer 30 then processes, in a step 1102, the data 200. For instance, in various embodiments, the computer 30 carries out an optional pre-processing in a step 1104.

For instance, in various embodiments, the computer 30 may generate data NGS′ by normalizing, in step 1104, each parameter NGS of the dataset 200. In particular, in various embodiments, the computer normalizes the matrix 200 by 10⁶, so that the data NGS' of each patient PR, i.e., each row, adds up to 1 instead of 10⁶, as occurs for the standard TPM values.

In various embodiments, the computer 30 may also generate pre-processed data DFS' by processing the data DFS that indicate a disease-free survival time, for example to converting dates 2002 into a corresponding number of days with respect to the date of treatment.

In addition or as an alternative, the computer 30 may scale, preferably by means of a nonlinear function, the data NGS or NGS′. For instance, in various embodiments, the computer 30 scales the data NGS' with a logarithmic function, such as a binary logarithm (log 2).

As explained previously, the matrix 200 (or the matrix resulting from the pre-processing 1104) comprises a number of variables p (e.g., the columns) much higher than the number n of reference patients PR (e.g., the rows), with n<<p. From a mathematical standpoint, the matrix 200 (or the matrix resulting from pre-processing 1104) is hence written in a redundant form, since it can be easily demonstrated that the rank of the columns of the matrix is equal to the rank of the rows. Consequently, in various embodiments, the computer is configured for generating in a step 1106 a matrix 202 by projecting the matrix 200 (or preferably the matrix resulting from pre-processing 1104) into an n-dimensional subspace via a principal-component analysis (PCA). PCA and its variants are well known to the person skilled in the art. For instance, for this purpose, it is possible to cite the book by T. Jolliffe, “Principal Component Analysis”, Springer Series in Statistics, Springer-Verlag, New York, 2002, ISBN 0-387-95442-2, the contents of which are incorporated for this purpose herein for reference.

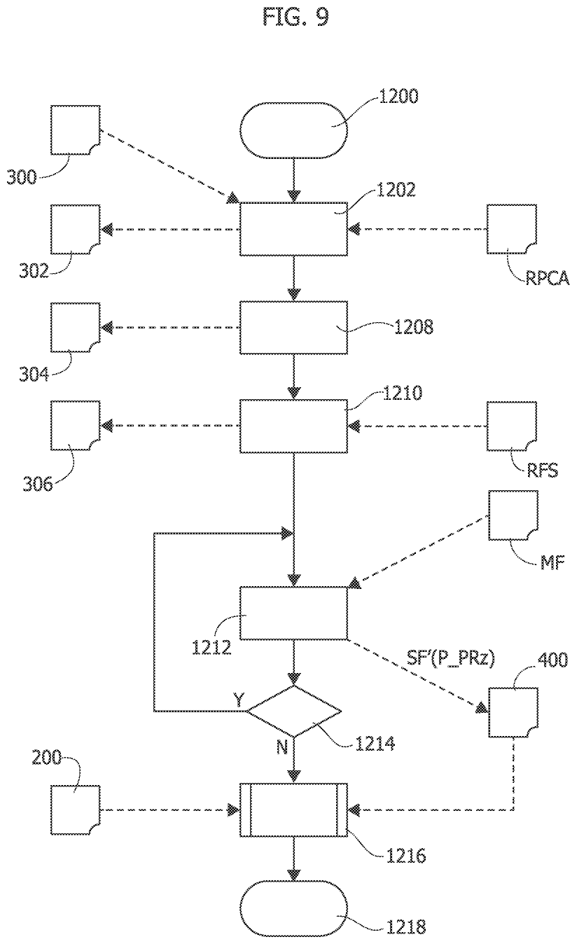

Consequently, as illustrated in FIG. 6, step 1106 provides a matrix 202 that comprises only n parameters PC, i.e., the so-called principal components. The matrix 202 is hence much smaller than the original one 200, albeit containing the same information, at the cost of having written the new feature n as combination, for example linear combination, of the original features p. This is not a problem from a mathematical standpoint, but could reduce the interpretability of the model since it becomes more difficult to set in relation the final result with the genes and understand which genes contribute to the forecasts.

In various embodiments, the computer 30 stores, in step 1106, also the mapping rules RPCA used for generating the n parameters PC of a given patient PR as a function of the respective p variables NGS (or the respective pre-processed variables), for example the respective function of combination, e.g., linear combination, of the respective p variables.

Consequently, in various embodiments, the dataset 202 comprises a list of n reference samples/patients PR, where each reference element/patient PR of the list comprises n variables PC and the respective data DFS (or DFS′).

In a traditional machine-learning method, such as the solution described in the article by Tong Li, the above dataset 202 (or directly the dataset 200) could then be used to solve a conventional regression problem, in which the model seeks to estimate directly the survival time DFS (or a similar value) of a patient, for example using the data PC as input of an artificial neural network, where the neural network supplies at output an estimate of the survival time DFS.

Instead, in various embodiments of the present disclosure, the machine-learning method receives at input the data of a pair of patients, and the method is trained to estimate which member of the pair has a longer survival time.

Consequently, in various embodiments, the computer 30 generates, in a step 1108, a list 204, generating pairs of patients by combining all the reference patients PR with one another. For instance, as shown in FIG. 7, the computer may generate pairs of patients PR₁_PR₂, PR₁_PR₃, PR₁_PRₙ, by combining the first patient PR₁ with all the other patients PR₂, . . . PRₙ, pairs of patients PR₂_PR₁, PR₂_PR₃, PR₂_PRₙ, by combining the second patient PR₂ with all the other patients PR₃, . . . PRₙ, etc. Hence, in various embodiments, the list 204 comprises n(n−1) elements, denoted hereinafter as elements PRₓ_PR_{y}, where each element PRₓ_PR_{y} comprises the data of respective features F obtained via the combination of the data PC (when the data 202 of step 1106 are used), of the data NGS' (when the data of step 1104 are used), or of the data NGS (when the original data 200 are used) of a respective first patient PRₓ and a respective second patient PR_{y}.

For instance, in various embodiments, the number of the features F corresponds to the number n of the variables/principal components PC, and the value of each feature Fᵢ, with 1≤i≤n, for a given pair of patients PRₓ and PR_{y}, i.e., Fᵢ(PRₓ_PR_{y}), is obtained by combining the value of the respective variable PCᵢ of the patient PR_{y}, i.e., PCᵢ(PR_{y}), with the value of the respective variable PCᵢ of the patient PR_{y}, i.e., PCᵢ(PR_{y}). For instance, in various embodiments, the computer 30 uses a measurement of distance between the values PCᵢ(PR_{y}) and PCᵢ(PR_{y}). For instance, in various embodiments, the computer is configured for computing the value of each feature Fᵢ(PRₓ_PR_{y}) via a so-called z-score, where the computer computes the value of the feature Fᵢ(PRₓ_PR_{y}) as the difference between the respective values PCᵢ(PR_{y}) and PCᵢ(PR_{y}), for example Fᵢ(PRₓ_PR_{y})=PCᵢ(PRₓ)−PCᵢ(PR_{y}). Then, the computer 30 computes the standard deviation of the feature Fᵢ and normalizes each value Fᵢ by dividing the value Fᵢ by the respective standard deviation. However, also other measurements of distance may be used, such as Euclidean distance, Minkowski distance, etc.

In various embodiments, the new list/matrix 204 hence comprises n(n−1) elements PRₓ_PR_{y}, with 1≤x≤n, 1≤y≤n and x≠y, where each element PRₓ_PR_{y} comprises a set of features F, which comprises n features Fᵢ, with 1≤i≤n, where each feature identifies a measurement of distance between the respective values PCᵢ(PRₓ) and PCᵢ(PRₓ) of the patients PRₓ and PR_{y}.

Moreover, the computer 30 determines, for each pair of patients PRₓ_PR_{y}, also a survival index SF, which indicates which of the two patients PRₓ or PR_{y} has a longer disease-free survival time, for example by setting the value SF at −1 (or 0) in the case where the patient PRₓ has a longer disease-free survival time or at +1 in the case where the patient PR_{y} has a longer disease-free survival time. In particular, for this purpose, the computer 30 may compare the disease-free survival time of the patient PRₓ, i.e., the data DFS(PRₓ) or DFS′(PRₓ), with the disease-free survival time of the patient PR_{y}, i.e., the data DFS(PR_{y}) or DFS′(PR_{y}).

In this context, it should be noted that the data DFS (or DFS′) of two patients PRₓ and PR_{y} should hence be comparable. However, in the case where the dataset also comprises censored patients, not all the pairs PRₓ_PR_{y} are effectively comparable, since two patients PRₓ and PR_{y} form a comparable pair only when they are both not censored or when only the one who has survived longer is censored. Consequently, in step 1108, the computer 30 may remove these elements, and the final number of elements in the list 204 may even be smaller. Hence, in general, the list 204 may comprise even just a subset of the pairs PRₓ_PR_{y}, where each pair PRₓ_PR_{y} has associated a respective set of features F determined as a function of the data of the respective patients PRₓ and PR_{y}, and a respective index SF that indicates which of the two patients PRₓ and PR_{y} has a longer disease-free survival time.

In various embodiments, after reorganization of the data in step 1108, the computer 30 may then train a machine-learning algorithm in such a way as to estimate the value of the index SF as a function of the values of the features F calculated for two patients. For instance, in various embodiments, the computer 30 may proceed to an optional step 1110 of feature selection, where the computer generates a list 206 that comprises a set of features EF, where the computer 30 generates the features EF by selecting a subset of the features F; namely, the number of the features EF is smaller than the number of the features F, for example smaller than n in the case where the data PC are used (see also FIG. 8).

In general, a very large number of feature-selection methods are known. For instance, with reference to the specific application, the inventors have noted that a LASSO model is particularly useful. This method is well known to the person skilled in the art and described, for example, in the article by Tibshirani, Robert, “Regression Shrinkage and Selection via the Lasso”, Journal of the Royal Statistical Society, Series B (methodological), 1996, Wiley, 58 (1): 267-88, DOI:10.1111/J.2517-6161.1996.TB02080.X, the contents of which are incorporated herein for reference. For instance, by training a LASSO model in step 1110, the computer 30 may generate a list 206 by removing from the list 204 all the features Fᵢ the LASSO coefficients of which are equal to 0. However, the LASSO method may be replaced with other feature-selection methods, for example one or more methods chosen from the following list:
wrapper methods, for example using a recursive feature elimination, a forward feature selection, or a backward feature selection;
filters, for example based upon the chi-squared method, Pearson correlation, relief or Fisher score; or
embedded methods, for example based upon decision trees, the so-called random forests, sparse multinomial logistic regression, automatic relevance determination, or regularization-based methods, for example ridge and elastic net.

For instance, for this purpose it is possible to cite the article by A. Jovi\tilde{c}, et al. “A review of feature selection methods with applications”, May 2015, 38th International Convention on Information and Communication Technology, Electronics and Microelectronics (MIPRO), DOI:10.1109/MIPRO.2015.7160458, the contents of which are incorporated herein for reference.

Consequently, in a step 1112, the computer 30 may train a classifier configured for estimating the value of the survival index SF as a function of the values of the features EF (or even directly of the features F) determined for two patients using for this purpose the dataset 206 (or the dataset 204 when step 1110 is omitted).

For instance, in various embodiments the computer 30 uses a k-nearest neighbour (k-NN) classifier. However, the computer 30 may also use one or more other classifiers, such as artificial neural networks (for example, a network of the multilayer-perceptron type), support vector machines, Gaussian process classifiers, decision trees, random forests, quadratic discriminant analysis, or Gaussian naive Bayes classifiers. Also these machine-learning methods are well known to the person skilled in the art, and it is possible to cite, for example, Abdullah, Siti, et al. “Support Vector Machine, Multilayer Perceptron Neural Network, Bayes Net and k-Nearest Neighbor in Classifying Gender using Fingerprint Features”, International Journal of Computer Science and Information Security, (Vol. 14 No. 7), or Thanh Noi, P., et al. “Comparison of Random Forest, k-Nearest Neighbor, and Support Vector Machine Classifiers for Land Cover Classification Using Sentinel-2 Imagery. Sensors” 2018, 18, 18. DOI:10.3390/s18010018, the contents of which are incorporated herein for reference.

Consequently, by the end of step 1112 the computer 30 has trained the classifier, i.e., determined the respective parameters MF of the classifier, and the learning process terminates in an end step 1114. As explained previously, the specific choice of the algorithm of feature selection 1110 and machine learning 1112 is not particularly important for the purposes of the present disclosure and may also vary according to the specific dataset 200 used and the specific desease considered. In fact, the present description regards above all the reorganization of the dataset 200 into pairs of patients and the estimation of an index SF that indicates which of two patients has a longer disease-free survival time.

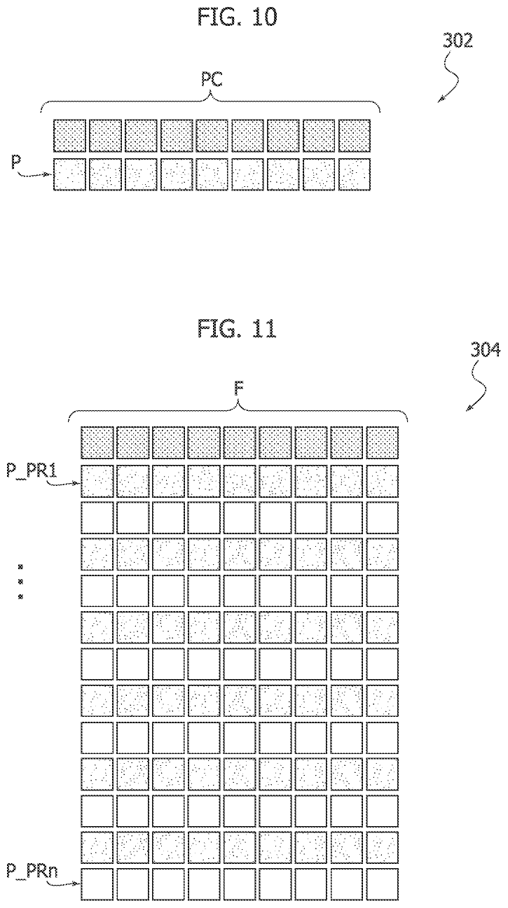

Next, as illustrated in FIG. 9, once step 1200 has been started, the computer 30 receives the data 300, in particular the respective data NGS, of a given patient P, and carries out operations similar to the ones carried out in steps 1102-1110.

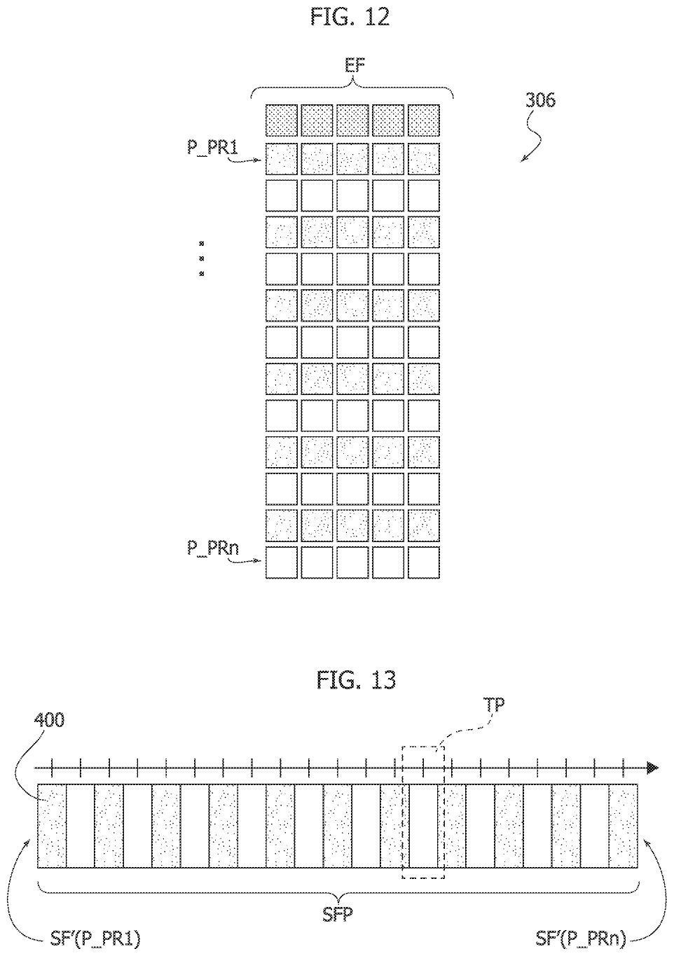

For instance, in various embodiments, the computer 30 may pre-process the data 300, in a step 1202, to generate pre-processed data 302. For instance, as in step 1204, the computer 30 may generate data NGS' by normalizing in step 1104 each parameter NGS of the dataset 300, and/or scale the data NGS or NGS' of the dataset 300. Moreover, in various embodiments, the computer 30 may generate a dataset 302 by computing variables PC as a function of the data NGS (or of the pre-processed data using the mapping rules RPCA generated in step 1106). Consequently, as shown in FIG. 10, the dataset 300 of the patient P may be converted into a dataset 302 that comprises n variables PC determined as a function of the data NGS.

As explained before, the classifier should receive at input the values determined for pairs of patients. Consequently, in a step 1208, the computer 30 generates a list 304, creating pairs of patients by combining this time the patient P with all the reference patients PR.

For instance, as illustrated in FIG. 11, the computer may generate pairs of patients P_PR₁, P_PR₂, . . . . P_PRₙ. Hence, in various embodiments the list 304 comprises n elements, denoted hereinafter as elements P_PR_{z}, with 1≤z≤n, where each element P_PR_{z} comprises the data of the respective features F obtained via combination of the data PC (when the data 302 are used), of the data NGS' (when the pre-processed data are used), or of the data NGS (when the original data 300 are used) of the patient P and a respective patient PR_{z}. For instance, in various embodiments, the value of each feature Fᵢ(P_PR_{y}) is obtained by combining the value of the respective variable PCᵢ of the patient P, i.e., PCᵢ(P), with the value of the respective variable PCᵢ of the patient PR_{z}, i.e., PCᵢ(PR_{z}), for example using the measurement of distance used in step 1108. Consequently, as compared to the list 204, the list 304 does not comprise the index SF, but only the values of the features F.

In the case where a feature-selection step 1110 has been used, the computer 30 may then generate, in a step 1210, a list 306 that comprises a subset of features EF by selecting a subset of the features F using the selection rules RFS (see also FIG. 12). In general, the computer 30 could also compute directly only the values of the features EF.

Consequently, the verification dataset 306 (or directly 304) does not comprise just a single element, but a list of elements, where each element P_PR_{z} comprises the respective values of the features EF.

In various embodiments, the computer then selects an element P_PR_{z} of the list 306 (or 304), for example the element P_PR₁, and uses the trained classifier, i.e., the parameters MF, to estimate an index SF′(P_PR_{z}) as a function of the respective values of the element selected P_PR_{z}, and saves the estimated index SF′(P_PR_{z}) in a list 400.

The computer 30 may then verify, in a step 1214, whether the list 306 (or directly 304) comprises at least one further element to be classified. In the case where the list 306 (or directly 304) comprises at least one further element (output “Y” from verification step 1214), the computer 30 selects a next element and returns to step 1212 for estimating the respective index SF′.

Consequently, via the steps 1212 and 1214, the computer 30 estimates, for each element of the list 304 (or directly 306), a respective index SF′, i.e., indices SF′(P_PR₁), SF(P_PRₙ) for the elements P_PR₁, P_PRₙ. However, these indices SF′ provide only an estimate that indicates whether the disease-free survival time of the patient P is longer or shorter than the disease-free survival time of the respective patient PR_{z}.

Consequently, in the case where the computer 30 has processed all the elements in the list, i.e., in the case where the list 306 (or directly 304) does not comprise further elements (output “N” from the verification step 1214), the computer 30 may proceed to a step 1216 where it estimates the disease-free survival time of the patient P as a function of the estimated indices SF′ and the disease-free survival times of the reference patients, for example as saved in the dataset 200 (or in the pre-processed dataset 202), and the process terminates in an end step 1218.

Consequently, as illustrated also in FIG. 13, step 1216 receives a list 400 that comprises the sequence of the indices SF′(P_PR₁), SF′(P_PRₙ) for the elements P_PR₁, . . . , P_PRₙ.

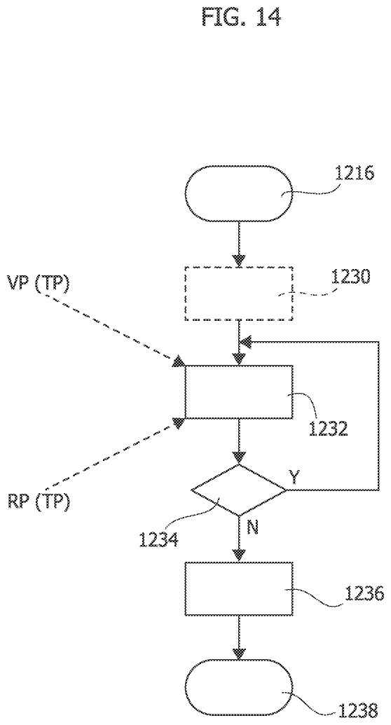

FIG. 14 shows a possible embodiment of step 1216.

In particular, once step 1216 has been started, the computer 30 may optionally order, in a step 1230, the list 400 according to the survival time of the reference patients PR of the respective pairs SF′(P_PR₁), . . . , SF′(P_PRₙ). In general, this step is purely optional because the list 306 (or possibly already the list 304) could already be ordered. For instance, in various embodiments, the computer is configured to order, for example in step 1102, the reference patients PR according to their survival time; for example, the patient PR₁ may be the patient with the shortest survival time, and the patient PRₙ may be the patient with the longest survival time, or vice versa. Consequently, in this way, also the lists 304, 306, and 400 are already ordered.

As a result, in various embodiments, the list 400 comprises a pattern SFP that corresponds to the sequence of the orderly indices SF′(P_PR₁), . . . , SF′(P_PRₙ), where the first index SFP(1) is associated to the reference patient with the shortest survival time (or alternatively the longest survival time), e.g., SF′(P_PR₁), and the last index SFP(n) is associated to the reference patient with the longest survival time (or alternatively shortest survival time), e.g., SF′(P_PRₙ). For instance, hereinafter it is assumed that each index SFP/SF′ is set at a first value, e.g., 0, when the survival time of the patient P is expected to be longer than the survival time of the patient PR_{z} and is set at a second value, e.g., 1, when the survival time of the patient P is expected to be shorter than the survival time of the patient PR_{z}. For instance, hereinafter it is assumed that the list 400 corresponds to the following pattern SFP of orderly indices SF′(P_PR₁), . . . , SF′(P_PRₙ):
SFP=“010 0000010 000001101101011101111111110111”

As may be noted, tendentially the classifier is not perfect, and consequently the sequence SFP does not comprise a clear separation between a sequence of 0's (first value) and a sequence of 1's (second value), but comprises also reversed values. Consequently, the computer 30 should take into consideration these reversed values.

In particular, in various embodiments, the computer 30 selects a test position TP, for example the first position, i.e., TP=1, and computes a congruence index CI(TP) for the test position TP.

For this purpose, in various embodiments, the computer 30 is configured for extracting from the sequence SFP for the position TP a respective pattern to be verified VP(TP). For instance, in various embodiments, the pattern VP(TP) comprises the entire sequence SFP of orderly indices SF′(P_PR₁), SF′(P_PRₙ), i.e., each pattern VP(TP) comprises the sequence SFP, for example:

VP(TP)=SFP=“010 0000010 000001101101011101111111110111”

Consequently, in a step 1232, the computer 30 may compare the current pattern VP(TP) with a reference pattern RP(TP) that corresponds to the (ideal) sequence of the values SF′ in the case where the position TP corresponds to the position where switching occurs between the sequence of 0's (first value) and the sequence of 1's (second value).

For instance, for the case provided by way of example (VP(TP)=SFP), the patterns RP(TP) may be the following:
RP(1)=“0111111111111111111111111111111111111111”,
RP(2)=“0011111111111111111111111111111111111111”,
. . .
RP(n)=“0000000000000000000000000000000000000000”.

For instance, in various embodiments, the computer 30 determines the congruence index CI(TP) through a measurement of similarity between the pattern VP(TP) and the reference pattern RP(TP), for example by counting the number of values that are the same for the patterns VP(TP) and RP(TP), possibly normalizing the results by n. Consequently, in this case, the best position corresponds to the position TP with the respective highest congruence index CI(TP). Alternatively, the computer may compute, in step 1232, an (in) congruence index CI(TP) through a measurement of distance between the pattern VP(TP) and the reference pattern RP(TP), for example by computing a sum of the absolute differences SAD, possibly normalizing the results by dividing the measurement of distance SAD by n. Consequently, in this case, the best position corresponds to the position TP with the lowest respective congruence index CI(TP).

Consequently, once the computer 30 has calculated the congruence index CI(TP) for the current position TP, it may verify, in a step 1234, whether at least one further position TP has to be verified, for example TP<n. In the case where at least one further position TP has to be verified (output “Y” from the verification step 1234), the computer 30 selects the next position and returns to step 1232.

Instead, in the case where all the positions TP have been verified (output “N” from the verification step 1234), the computer 30 proceeds to a step 1236 to select the position TP* that corresponds to the best position, i.e., the position TP with the lowest (or highest) respective congruence index CI(TP), and the process terminates in an end step 1238. Consequently, in general, steps 1232 and 1234 may be implemented also directly in steps 1212 and 1214.

For instance, using a normalized congruence index CI, the computer 30 could compute the following congruence indices CI(TP):
CI(1)=0.575
CI(2)=0.550
. . .
CI(14)=0.800
CI(15)=0.825,
CI(16)=0.800,
. . .

Consequently, in this case, the computer 30 could select the position TP* with the congruence index CI(TP) having the maximum value, for example the index TP=15 with CI(15)=0.825. In general, the computer could also take into consideration one or more adjacent congruence (or incongruence) values, for instance computing, in step 1236, a global congruence index CCI(TP) by adding to the congruence index CI(TP) one or more previous and/or subsequent congruence indices; for example,

CCI(TP)=CI(TP−1)+CI(TP)+CI(TP+1)

and then selecting the position TP* with the respective global congruence index CCI(TP) having the maximum value (or likewise a global incongruence index CCI(TP) having the minimum value). In general, the global index CCI(TP) may thus be obtained by filtering the index CI(TP), for example via a moving average, possibly weighted.

In the embodiment considered, the first pattern RP(1) hence corresponds to the ideal sequence when the patient P has a survival time between that of the first reference patient PR₁ and that of the second reference patient PR₂. Instead, the last pattern RP(n) corresponds to the ideal sequence when the patient P has a survival time longer than that of the last patient PRₙ. Consequently, in various embodiments, to verify also the situation in which the patient P has a survival time shorter than that of the first patient PR₁, the computer may also verify, in step 1232, a further pattern RP(0) that is compared with the pattern VP(1); for example:
RP(0)=“111111111111111111111111111111111111111111” In general, the patterns VP(TP) may even correspond just to a subset of the pattern SFP. For instance, in various embodiments, each pattern SFP(TP) comprises k orderly indices SF, where the parameter k is smaller than, or corresponds to, the number n. Preferably, the parameter k is equal for all the sequences VP. For instance, in the case where the parameter k is smaller than the number n, the parameter k is in any case preferably higher than 10, preferably higher than 50, for example between 100 and 500. Consequently, the pattern SFP(TP) comprises the index SF′ at the position TP, i.e., SF′(P_PR_{TP}), plus a plurality of indices SF′ before and/or after the position TP. For instance, by (virtually) adding a sequence of 0's (first value) at the start of the sequence SFP and a sequence of 1's (second value) at the end of the sequence SFP, a sequence VP(TP) may be determined by selecting a given number of indices SF′ before the position TP, the index SF′ at the position TP, and a given number of indices SF′ after the position TP, for example always selecting k=13 indices SF′:
VP(1)=“0000000100000”
VP(2)=“0000001000000”
VP(3)=“0000010000001”
. . .
VP(n)=“1111111110111”

Consequently, also in this case, the computer may compute, in step 1232, for each pattern VP(TP) a respective congruence (or incongruence) index CI(TP) by comparing the pattern VP(TP) with a respective pattern RP(TP) that corresponds in this case to the (ideal) sequence of the k values SF′ in the case where the position TP corresponds to the position where switching occurs between the sequence of 0's (first value) and the sequence of 1's (second value); for example:
VP(1)=VP(2)==VP(n)=“0 000001111111” Also in this case, the computer 30 may then select, in step 1236, the position TP* with the best index CI(TP) or CCI(TP).

In general, the sub-sequences VP(TP) may be chosen even differently, using, however, the pattern RP(TP) that corresponds to the (ideal) sequence of the k values SF′ in the case where the position TP corresponds to the position where switching occurs between a sequence of 0's (first value) and a sequence of 1's (second value). In fact, it is sufficient for the computer 30 to be able to compute (in step 1232), for each position TP of a plurality of positions TP of the list 400, a respective parameter TP that indicates the probability of the patient P having a disease-free survival time DFS longer than the disease-free survival time DFS of the reference patient PR in the position TP, but shorter than the disease-free survival time DFS of the reference patient PR in the next position TP+1.

Consequently, once the computer 30 has selected the position TP*, it may estimate the disease-free survival time DFS(P) of the patient P with the disease-free survival time DFS(PR_{TP}*), or in any case a value comprised between the disease-free survival time DFS(PR_{TP}*) and the disease-free survival time DFS(PR_{TP*+1}). Consequently, in various embodiments, the computer 30 may determine, for example using the list 200, the survival time of the patient PR at the position TP*, i.e., DFS(PR_{TP*}) and display, for example on a screen of the computer, the time DFS(PR_{TP*}) as prognosis. Optionally, the computer 30 may also determine, for example using the list 200, the survival time of the patient PR at the position TP*+1, i.e., DFS(PR_{TP*+1}), and display also this time.

In various embodiments, the computer 30 may determine, in step 1216, also a maximum recommended follow-up time as a function of the time DFS(P), for example by dividing the estimated time by a given coefficient, for instance chosen in the range between 1 and 5.

Of course, without prejudice to the underlying principles of the invention, the details of construction and the embodiments may vary widely with respect to what has been described and illustrated herein purely by way of example, without thereby departing from the scope of the present invention as this is defined in the annexed claims.

1. A method for prognosis of a given disease following a therapeutic treatment including:
during a training phase:
receiving a dataset comprising a plurality of reference patients having performed a therapeutic treatment of said given disease, wherein said dataset comprises for each reference patient of said plurality of reference patients respective omics data and data indicating a disease-free survival time of the respective reference patient after the respective therapeutic treatment of said given disease;
generating for each reference patient respective pre-processed data via a principal component analysis of the respective omics data, and storing the respective mapping rules used to generate said pre-processed data as a function of the respective omics data;
generating a training dataset by forming reference patient pairs between a respective first reference patient and a respective second reference patient and determining for each reference patient pair:
a respective feature set calculated via a distance measure between the pre-processed data of the respective two reference patients, and
a respective index indicating which of the respective two reference patients has a longer disease-free survival time;
training a classifier configured to estimate said index as a function of the respective feature set calculated for the pre-processed data of two patients by using said training dataset; and
during a prognosis phase:
receiving omics data of a patient;
generating for said patient respective pre-processed data by using said mapping rules;
generating a verification dataset by forming pairs of patients by combining said patient with a plurality of reference patients and determining for each pair of patients a respective feature set calculated by said distance measure between the pre-processed data of said patient and the pre-processed data of the respective reference patient;
estimating for each pair of patients a respective index by providing the feature set of the respective pair of patients to said classifier;
generating a list of said estimated indices, wherein said list is sorted according to the disease-free survival time of the reference patients of the pairs of patients;
calculating for each position of a plurality of positions of said list a respective parameter indicating the probability that said patient has a disease-free survival time being greater than the disease-free survival time of the reference patient in said position, but smaller than the disease-free survival time of the reference patient in the next position, and
selecting the position for which the respective parameter indicates the highest probability, and estimating the disease-free survival time of said patient with a value between the disease-free survival time of the reference patient in said selected position and the disease-free survival time of the reference patient in the position following said selected position.

2. The method according to claim 1, comprising setting said index to a first value to indicate that the respective first reference patient has a disease-free survival time being greater than the disease-free survival time of the respective second reference patient, and to a second value to indicate that the respective first reference patient has a disease-free survival time being smaller than the disease-free survival time of the respective second reference patient.

3. The method according to claim 2, wherein said calculating for each position a respective parameter comprises:
generating for each position a respective pattern to be verified by selecting the index of said list at said position, and a first number of indexes of said list before said position and/or a second number of indexes of said list after said position,
obtaining for each pattern to be verified a respective reference pattern, wherein said reference pattern corresponds to a pattern of indices in case said position corresponds to the position at which occurs the switching between a sequence of said first number of said first value and a sequence of said second number of said second value takes place, and
calculating the value of said parameter associated with said position by means of a similarity measure or a distance measure between the respective pattern to be verified and the respective reference pattern.

4. The method according to claim 2, wherein each pattern to be verified corresponds to the sequence of said estimated indices of said list, and the reference pattern associated with said position has set all indices up to said position to said first value, and all indices after said position to said second value.

5. The method according to claim 1, wherein said generating for each reference patient respective pre-processed data comprises normalizing said omics data, and/or scaling said omics data via a non-linear function.

6. The method according to claim 1, wherein said omics data comprises transcriptomic data obtained via Next Generation Sequencing.

7. The method according to claim 1, wherein said training a classifier comprises:
selecting a subset of said features via a feature selection method, such as LASSO, and training a classifier configured to estimate said index as a function of the respective subset of features calculated for the pre-processed data of two patients using said training dataset.

8. The method according to claim 1, wherein said classifier comprises at least one of: a k-nearest neighbor classifier, an artificial neural network, such as a multi-layer perceptron type network, a support vector machine, a gaussian process classifier, decision trees, random forests, quadratic discriminant analysis, and/or Naïve Bayes gaussian classifiers.

9. A device configured to implement the method according to claim 1.

10. A computer-program product that can be loaded into the memory of at least one processor and comprises portions of software code for implementing the steps of the method according to claim 1.
